(19) United States
(12) Patent Application Publication
(10) Pub. No.: US 2009/0227901 A1
(43) Pub. Date: Sep. 10, 2009

(54) CATHETER GUIDEWIRE AND METHOD FOR MANUFACTURING SAME

(75) Inventor: Eugen Hofmann, Zurich (CH)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: Biotronik VI Patent AG, Baar (CH)

(21) Appl. No.: 12/398,732

(22) Filed: Mar. 5, 2009

(30) Foreign Application Priority Data
Mar. 5, 2008 (DE) 10 2008 012 743.4

(51) Int. Cl. A61M 25/01

(52) U.S. Cl. 600/585

(57) ABSTRACT

A catheter guidewire (1), preferably for cardiovascular procedures, having a distal end (2) and a proximal end (3) and a one-piece core (10) extending essentially from the distal end to the proximal end. The one-piece core has twisted fibers, preferably glass fibers, which are embedded in the basic material of the core. Also disclosed is a method for manufacturing a catheter guidewire.

### PRIORITY CLAIM

This patent application claims priority to German Patent Application No. 10 2008 012 743.4, filed Mar. 5, 2008, the disclosure of which is incorporated herein by reference in its entirety.

### FIELD

The present disclosure relates to a catheter guidewire, preferably for cardiovascular procedures, having a distal end and a proximal end and a one-piece core extending essentially from the distal end to the proximal end. The present disclosure also relates to a method for manufacturing such a catheter guidewire.

### BACKGROUND

When catheters are used, e.g., as part of cardiovascular procedures, the catheter is positioned with the help of a guidewire previously inserted into the patient's body. The distal tip of a catheter guidewire is usually designed to be very flexible so that the distal tip can be bent and twisted to be able to advance the tip to the desired location in the patient's body, e.g., inside a blood vessel.

International Patent Publication No. WO 98/42268 discloses a guidewire having a core of a glass body. The glass core is made of fiberglass, silicon or quartz, for example, and is surrounded by a coating. The glass core with the coating that serves to reinforce the glass core extends essentially over the length of the guidewire and is surrounded by a plastic sheathing that is attached to the coating on the glass body with an adhesive. The outer plastic sheathing is provided to maintain the physical integrity of the guidewire even if the glass core should break. For this purpose, the sheathing has reinforcing fibers, for example, such that the sheathing may be braided with the reinforcing fibers. The reinforcing fibers may also be arranged in a polymer matrix. For example, the material for the reinforcing fibers may be carbon, aramid or glass.

German Patent Application No. 10 2004 028 367 discloses a catheter guidewire whose elongated core is designed in one piece and is made of polyether ether ketone (hereinafter referred to as ) plastic continuously from the proximal end to the distal end. The core is designed with a taper in the end area of its distal end so the diameter tapers in comparison with the diameter at the proximal end. The guidewire has a coating in which a magnetic resonance tomography (hereinafter referred to as MRT) contrast medium is provided. Elements of the lanthanide group, such as gadolinium or dysprosium, may be used as the MRT contrast medium. The MRT contrast media serve to monitor the advance of the guidewire, e.g., via the blood vessels, up to the target site of the treatment. This allows monitoring by means of a magnetic resonance tomography device (hereinafter also referred to as an MRT scanner) which supplies imaging information in a stress-free manner for the patient.

However, the known guidewire with a PEEK core has the disadvantage that the guidewire is not rigid enough for some applications. In particular, such a PEEK guidewire can be permanently deformed in a spiral after storage due to the so-called “creep” of the plastic so that the linearity of the guidewire as required for insertion of the guidewire no longer prevails. In this case, the guidewire can be used clinically only to a limited extent. Furthermore, the particles in the polymer coating provide only diffuse and, therefore, inaccurate visualization in the MRT scanner.

### SUMMARY

The present disclosure describes several exemplary embodiments of the present invention.

One aspect of the present disclosure provides a catheter guidewire, comprising a) a distal end; b) a proximal end; and c) a one-piece core extending essentially from the distal end to the proximal end, wherein the core has twisted fibers embedded in the material structure of the core.

Another aspect of the present disclosure provides a method for manufacturing a catheter guidewire having a distal end, a proximal end, and a one-piece core extending essentially from the distal end to the proximal end, wherein the core has twisted fibers embedded in the material structure of the core, the method comprising a) pultruding the fibers with the basic material of the core; b) twisting the fiber-reinforced material obtained in step a) with axial pulling and a heat treatment; c) grinding the twisted fiber-reinforced material to the desired diameter of the core; and d) applying a first coating to the fiber-reinforced material of the core.

One aspect of the present disclosure is thus to create a catheter guidewire whose linearity is preserved even after a storage time in a round package, where the guidewire is maintained in a circular arrangement, for example. With respect to flexibility and/or rigidity, the guidewire should also have very good properties. Furthermore, the required buckling resistance must be guaranteed. The present disclosure also provides a simple and inexpensive method for manufacturing such a guidewire.

The aspect described hereinabove is achieved by a catheter guidewire whose core has twisted fibers, preferably glass fibers, which are embedded in the basic material of the core.

The use of twisted fibers, such that each of the twisted fibers preferably extends over the total length of the core, the fibers preferably being twisted under axial tensile stress and under the influence of heat, ensures that the guidewire has the required rigidity because the twisted fibers have been twisted under axial stress. Because of the increased rigidity, the guidewire remains straight even after prolonged storage in a round package, for example.

In one preferred exemplary embodiment, the fiber component in the total material of the core which comprises the basic material and the fibers, amounts to at least approximately 30 vol %, preferably at least 40 vol %. The guidewire has such a good flexibility and/or rigidity that the movement of the guidewire in blood vessels is not hindered.

The material of the fibers preferably comprises SiO₂ such that, in an especially preferred exemplary embodiment, the fiber material is formed at least predominantly of Saint-Gobain quartz fibers with a diameter of at least 10 μm. Through such a choice of fiber material, the required buckling resistance of the catheter guidewire is achieved.

The core preferably has a first coating which sheaths the core such that the first coating preferably includes at least one material selected from the group consisting of polyurethane and polyamide.

The guidewire of the present disclosure is preferably designed so that the core in an end section on its distal end has a smaller diameter and the first coating on this end has a correspondingly increased thickness in comparison with the diameter of the core on the proximal end and the thickness of the first coating on the proximal end of the guidewire, respectively. As a result of the smaller diameter of the core and the increased thickness of the coating in the area of the distal end, the guidewire has a greater flexibility on the distal end. It is especially preferable for the ratio of the proximal diameter (diameter in the shaft section) to the distal diameter (diameter in the distal section) of the core to be approximately 2:1 to 5:1, preferably approximately 3:1.

In the exemplary embodiment, to achieve an optimum transition between the soft distal end of the guidewire and the stiff proximal end, the core has a conical section situated between the distal section of the core, which is arranged the greatest distance away distally, and the shaft section of the core, which is arranged at the closest distance proximally. The length of this conical section in the longitudinal direction amounts to at least approximately 50 mm, preferably at least approximately 100 mm. The end section of the core on its distal end is thus composed of the distal section and the conical section.

To allow passive visualization in an MRT scanner, the core has at least one marking of an MR-visible material. Such a marking is also referred to below as an MR marker.

The MR marker preferably has at least one material selected from the group consisting of iron, titanium, and the elements of the group of lanthanides, preferably gadolinium or dysprosium. In another exemplary embodiment, the core has several markings, preferably in the form of spheres, cylinders or hollow cylinders, preferably arranged at defined intervals, between the distal end and the proximal end. It is especially preferable if the total volume of the markings increases in the direction of the distal end.

In another exemplary embodiment, a plate-like control element, preferably made of stainless steel, is provided at least on the distal end of the core, preferably in the area of the distal section between the core and the first coating. By means of such an elongated flat wire, which may also serve as an MR marker and is arranged in the distal tip of the guidewire, the guidewire can be pre-bent and at the same time rendered visible in an MR scanner.

In another exemplary embodiment, a second coating which is hydrophilic, is arranged on the first coating, preferably not extending as far as the proximal end of the guidewire. The hydrophilic coating imparts good friction properties to the guidewire while the guidewire remains non-slip on the proximal end that does not have a hydrophilic coating.

One exemplary method for manufacturing a catheter guidewire according to the present disclosure comprises the following steps:
a) pultruding the fibers with the basic material of the core,
b) twisting the fiber-reinforced material obtained in step a) with axial pulling and a heat treatment,
c) grinding the twisted fiber-reinforced material to the desired diameter(s) of the core, and
d) applying the first coating to the fiber-reinforced material of the core.

The disclosed manufacturing method is simple and inexpensive and yields a guidewire having an increased stiffness so that its linearity is not lost even with prolonged storage. The grinding process in the method is of such a dimension in step c) that the diameter of the core on the distal end is reduced by grinding, forming a soft and atraumatic tip of the guidewire.

It is also preferable for the first coating to be extruded onto the core. The first coating, preferably comprising a plastic, protects the guidewire because the guidewire is still held together by the coating comprising the first coating in the event of buckling or breaking of the core and no parts of the core can be washed away in the blood vessel into which the guidewire is inserted.

In another exemplary embodiment, a second coating, which is hydrophilic, may also be applied to the first coating. This second coating does not extend to the proximal end of the guidewire but instead is arranged essentially on the distal end of the core and sheaths the first coating.

It is especially preferred if maximum ¼ revolutions per 10 mm length are produced in the longitudinal direction of the fibers by twisting in step b) so as not to reduce the buckling resistance of the catheter guidewire.

### BRIEF DESCRIPTION OF THE DRAWINGS

Additional goals, features, advantages and possible applications of the invention disclosed herein result from the following description of exemplary embodiments on the basis of the figures. All features which are described or illustrated in the drawings constitute the subject matter of the present disclosure either alone or in any combination, even independently of their summary in the individual claims.

Various aspects of the present disclosure are described hereinbelow with reference to the accompanying figures.

FIG. 1 shows a longitudinal section of a first exemplary embodiment of a guidewire of the present disclosure; and

FIG. 2 shows a longitudinal section of a second exemplary embodiment of a guidewire of the present disclosure.

### DETAILED DESCRIPTION

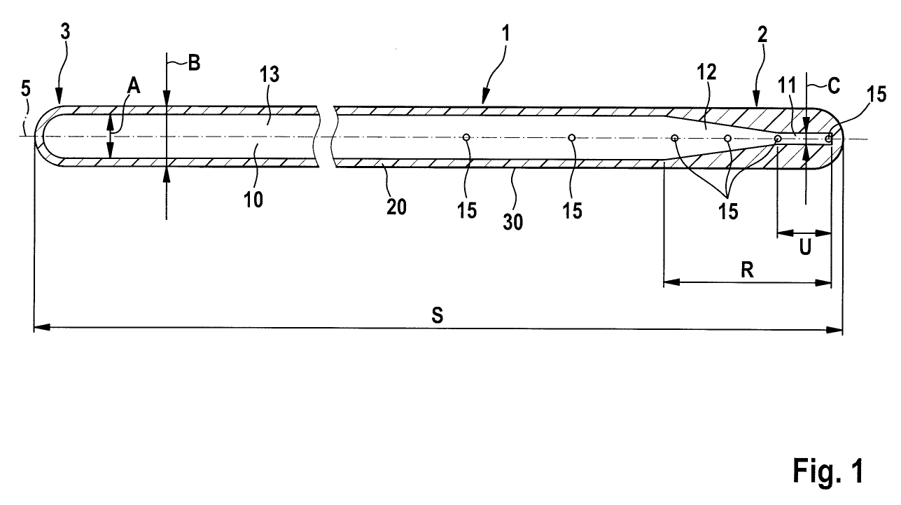

FIG. 1 shows a catheter guidewire 1 with a distal end 2 and a proximal end 3. The guidewire 1 has an essentially cylindrical core 10 whose basic material comprises PEEK plastic which is furnished with twisted Saint-Gobain quartz fibers with a diameter of approximately 10 μm. The core 10 extends in the longitudinal direction along the axis 5. The core 10 is sheathed by a plastic coating (first coating) 20, which is made of polyurethane (e.g., PU Pellethane Shore 90 A).

The coating 20 is in turn surrounded by a second coating 30, which is hydrophilic and extends over the length of the core, in particular, its distal end section, to the most proximal 20 cm of the length of the guidewire 1, for example. Before insertion, the hydrophilic coating has a thickness of 10 μm, for example. On insertion, the coating swells to a very great extent. The hydrophilic coating comprises a solution of photoreactive polyvinylpyrrolidone copolymer, for example.

The core 10 is composed of three sections: the distal section 11, the conical section 12 and the shaft section 13, which are arranged in this order one after the other as seen from the distal end of the core. In the distal section 11, the diameter C of the core 10 is the smallest, and in the shaft section 13, the diameter C of the core 10 is the greatest. In the conical section 12, there is a transition in the diameter of the core 10 from the lower value in the distal section 11 to the larger value in the shaft section 13.

In one exemplary embodiment, the guidewire has a length S of approximately 160 cm. The length R of the distal end section of the core comprising the distal section 11 and the conical section 12 amounts to approximately 120 mm. The length of the distal section 11 (also known as the floppy tip) is approximately 20 mm, and the length of the conical section 12 is approximately 100 mm. The diameter C of the core in the distal section is approximately between 150 and 250 μm, where the diameter C is constant over the entire distal section 11. The diameter A of the core in the area of the proximal end of the guidewire 1 (shaft section 13) amounts to approximately 0.57 mm. The outside diameter of the guidewire B in the area of its proximal end, where no second coating 30 is provided but instead there is only a core 10 and its first coating 20, amounts to approximately 0.88 mm.

MR markers 15 made of round stainless steel foil are applied to the core 10 for passive visualization in the MR scanner. The MR markers are arranged initially at a smaller distance and, more remotely from the distal end 2 of the guidewire, the MR markers are arranged at a greater distance. For example, six MR markers 15 are provided, arranged at a distance of 2 cm, starting from the distal end of the core, and two MR markers 15 are arranged at a distance of 4 cm. The MR markers 15 are punched from round metal foils with a diameter of 1.1 mm and are arranged around the core 10.

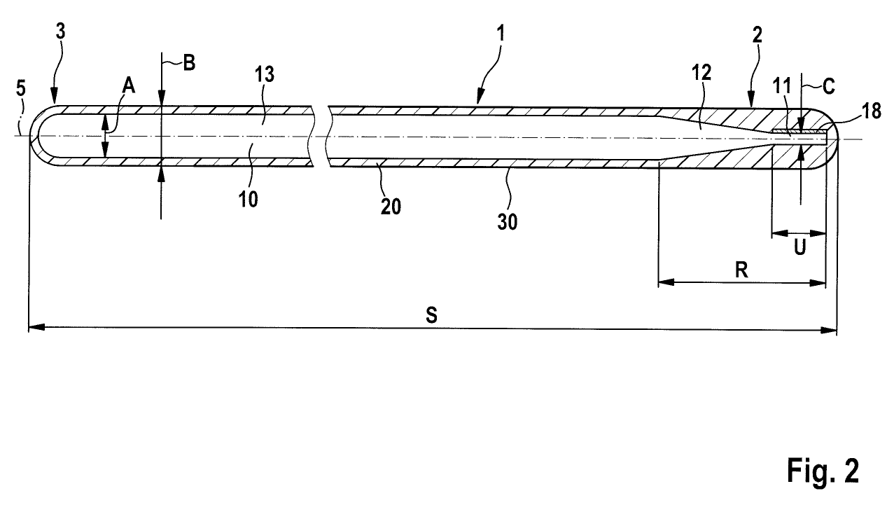

In another exemplary embodiment, which is illustrated in FIG. 2, between the core 10 and the first coating 20, the guidewire has a plate-like control element 18 in the form of a flat wire of stainless steel instead of the markings 15 in the distal section 11 of the core 10. This control element 18 serves, on the one hand, to move the distal tip 2 of the guidewire 1 and, on the other hand, to detect the position of the guidewire 1 in the MR scanner. The length of the control element 18 in the longitudinal direction of the catheter guidewire 1 amounts to approximately 20 to 50 mm.

All patents, patent applications and publications referred to herein are incorporated by reference in their entirety.

What is claimed is:

1. A catheter guidewire, comprising:
a) a distal end;
b) a proximal end; and
c) a one-piece core extending essentially from the distal end to the proximal end, wherein the core has twisted fibers embedded in the material structure of the core.

2. The catheter guidewire of claim 1, wherein each of the twisted fibers extends essentially over the length of the core such that the fibers are twisted under axial pulling and the influence of heat.

3. The catheter guidewire of claim 1, wherein the fiber component of the total material of the core amounts to at least approximately 30 vol %.

4. The catheter guidewire of claim 1, wherein the material of the fibers comprises SiO₂, such that the fiber material is formed at least predominantly from Saint-Gobain quartz fibers with a diameter of at least 10 μm.

5. The catheter guidewire of claim 1, wherein the basic material of the core comprises at least one plastic material.

6. The catheter guidewire of claim 1, wherein the core has a first coating such that the first coating contains at least one material selected from the group consisting of polyurethane and polyamide.

7. The catheter guidewire of claim 6, wherein the core has a smaller diameter on its distal end in one end section, and the first coating has an increased thickness accordingly, in comparison with the diameter of the core and the thickness of the first coating on the proximal end of the guidewire, respectively.

8. The catheter guidewire of claim 7, wherein the end section of the core has a distal section on its distal end and has a conical section, such that the conical section is arranged between the distal section and a shaft section of the core, and the conical section has a length of at least approximately 50 mm in the longitudinal direction.

9. The catheter guidewire of claim 1, wherein the core has at least one marking of an MR-visible material.

10. The catheter guidewire of claim 9, wherein the marking has at least one material selected from the group consisting of iron, titanium, and elements of the group of lanthanides.

11. The catheter guidewire of claim 9, wherein the core has a plurality of markings between the distal end and the proximal end such that the total volume of the markings increases in the direction of the distal end of the core.

12. The catheter guidewire of claim 1, wherein a leaf-shaped control element is provided between the core and the first coating in the area of the distal section.

13. The catheter guidewire of claim 1, further comprising a hydrophilic second coating disposed at least on the distal end of the core on the first coating.

14. A method for manufacturing a catheter guidewire having a distal end, a proximal end, and a one-piece core extending essentially from the distal end to the proximal end, wherein the core has twisted fibers embedded in the material structure of the core, the method comprising:
a) pultruding the fibers with the basic material of the core;
b) twisting the fiber-reinforced material obtained in step a) with axial pulling and a heat treatment;
c) grinding the twisted fiber-reinforced material to the desired diameter of the core; and
d) applying a first coating to the fiber-reinforced material of the core.

15. The method of claim 14, wherein the first coating is extruded onto the core.

16. The method of claim 14, further comprising a hydrophilic second coating applied to the first coating on the distal end of the core.

17. The method of claim 14, wherein the twisting is performed in step b) with maximum of ¼ revolutions per 10 mm length in the longitudinal direction.

18. The catheter guidewire of claim 1, wherein the fiber component of the total material of the core amounts to at least approximately 40 vol %.

19. The catheter guidewire of claim 7, wherein the end section of the core has a distal section on its distal end and has a conical section, such that the conical section is arranged between the distal section and a shaft section of the core, and the conical section has a length of at least approximately 100 mm in the longitudinal direction.
